(19) United States
(12) Patent Application Publication
(10) Pub. No.: US 2011/0201664 A1
(43) Pub. Date: Aug. 18, 2011

(54) Methods and Compositions for Treatment of Prostate Intraepithelial Neoplasia

(76) Inventors: Jack I. Zweig, New Rochelle, NY (US); Elizabeth Zweig, New Rochelle, NY (US)

(21) Appl. No.: 12/083,643

(22) PCT Filed: Oct. 14, 2006
(86) PCT No.: PCT/US2007/040307

§ 371 Date: Mar. 7, 2011

(51) Int. Cl. A61K 31/40; A61K 31/192; A61P 35/00

(52) U.S. Cl. 514/423; 514/569

(57) ABSTRACT

Provided herein are methods of treatment of prostate intraepithelial neoplasia (PIN) by administering bexarotene. Also provided are pharmaceutical compositions and dosing regimens.

### RELATED APPLICATIONS

This application claims priority to U.S. Provisional Application No. 60/726,753, filed Oct. 14, 2005 entitled “METHODS AND COMPOSITIONS FOR TREATMENT OF PROSTATE INTRAEPITHELIAL NEOPLASIA” to Jack I. Zweig. The disclosure of the above referenced application is incorporated by reference herein.

### FIELD

Provided herein are methods for treatment of prostate intraepithelial neoplasia (PIN) by administering bexarotene. Further provided are pharmaceutical compositions and dosing regimens for treatment of PIN.

### BACKGROUND

Prostate cancer is one of the most common malignancies diagnosed in men and is the most common cancer found in men older than 60 years. A third or more of all men older than 50 years have a latent form of prostate cancer that may progress to life-threatening prostate cancer. The number of men with latent prostate cancer is the same across all cultures, races, and ethnic groups, but the frequency of clinically active cancer is markedly different.

Environmental factors have been implicated in activating latent prostate cancer. If cancer can be identified in an early or latent stage, the neoplastic process may be reversed.

Prostatic intraepithelial neoplasia (PIN) has been identified as a precursor lesion to prostatic carcinoma. PIN refers to the precancerous end of a morphologic spectrum involving cellular proliferation within prostatic ducts, ductules, and acini. The term PIN refers to a variety of terms (e.g., intraductal hyperplasia, hyperplasia with malignant change, large acinar atypical hyperplasia, marked atypia, ductal-acinar dysplasia.)

PIN appears to precede cancer by more than 10 years, with a parallel age-related increase in the frequency of PIN and cancer. PIN has been found in 9% of men in the second decade of life, 22% of men in the third decade, and 40% of men in the fourth decade. By the time men reach age 80 years, the prevalence of PIN is 70%.

Treatment of PIN with 5-alpha reductase inhibitors, antiandrogens, or SERMs has been advocated as a form of cancer prevention but no clearly established protocol has been established. Therefore, there is a continuing need to develop treatments for PIN.

### SUMMARY

In one embodiment, provided herein are methods for treatment of prostate intraepithelial neoplasia by administering bexarotene or a pharmaceutically acceptable derivative thereof. In other embodiment, provided herein are methods for preventing prostate carcinogenesis by administering bexarotene or a pharmaceutically acceptable derivative thereof. Further provided are methods for reducing the risk of developing prostate cancer by administering bexarotene or a pharmaceutically acceptable derivative thereof. Also provided are methods for suppressing or inhibiting latent prostate cancer by administering bexarotene or a pharmaceutically acceptable derivative thereof. In certain embodiments, methods for treating prostate cancer by administering bexarotene or a pharmaceutically acceptable derivative thereof are provided. In other embodiments, methods for management of PIN by administering bexarotene or a pharmaceutically acceptable derivative thereof are provided. Further provided are methods for reducing the amount of precancerous precursors of prostate adenocarcinoma lesions by administering bexarotene or a pharmaceutically acceptable derivative thereof.

Also provided are articles of manufacture containing packaging material, bexarotene or pharmaceutically acceptable derivative thereof, and a label that indicates that bexarotene or pharmaceutically acceptable derivative thereof is used for treatment, prevention or amelioration of PIN or prostate cancer.

Further provided are kits for administration of appropriate amounts of bexarotene and other active ingredients to a patient. A typical kit includes a container containing a dosage form of bexarotene or a pharmaceutically acceptable derivative thereof and a container containing one or more other therapeutic agent(s) described elsewhere herein. Such other therapeutic agents include, but are not limited to anti-lipid agents, anti-hypothyroid agents, and chemopreventive agents.

### DETAILED DESCRIPTION OF THE INVENTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, “prostate intraepitheial neoplasia” or PIN refers to the precancerous end of a morphologic spectrum involving cellular proliferation within prostatic ducts, ductules, and acini.

As used herein, “prostate carcinogenesis” refers to the development of cancer starting from the very first phase, called the initiation phase, followed by the promotion phase, such as development of prostate intraepithelial neoplasis, and ending in the final phase of the disease, such as development of cancerous lesions, the progression phase.

As used herein, “chemoprevention” refers to the use of natural or synthetic substances to reduce the risk of developing cancer, or to reduce the chance that cancer will recur.

As used herein, a “chemopreventive agent” refers to any natural or synthetic substance that reduces the risk of developing cancer or to reduces the chance that cancer will recur. Exemplary chemopreventive agents include drugs, vitamins, diet, hormone therapy, or other agents known to possess chemopreventive activity.

As used herein “subject” is an animal, typically a mammal, including human, such as a patient. In certain embodiments, the patient is an adult male. Exemplary age range for the patient is described elsewhere herein.

As used herein, and unless otherwise specified, the term “pharmaceutically acceptable derivatives” include salts, esters, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, alkali metal salts, such as but not limited to lithium, potassium and sodium; alkali earth metal salts, such as but not limited to barium, calcium and magnesium; transition metal salts, such as but not limited to zinc; and other metal salts. Pharmaceutically acceptable esters include, but are not limited to, alkyl, alkenyl, alkynyl, aryl, heteroaryl, aralkyl, heteroaralkyl, cycloalkyl and heterocyclyl esters of acidic group.

As used herein, and unless otherwise specified, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein, and unless otherwise specified, the term “stereoisomer” encompasses all enantiomerically/stereomerically pure and enantiomerically/stereomerically enriched compounds of this invention.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder. Treatment also encompasses any pharmaceutical use of the compositions herein, such as use for treating PIN.

As used herein, amelioration of the symptoms of a particular disorder by administration of a particular pharmaceutical composition refers to any lessening, whether permanent or temporary, lasting or transient that can be attributed to or associated with administration of the composition.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, the terms “therapeutically effective amount” and “effective amount” of a compound mean an amount sufficient to provide a therapeutic benefit in the treatment, prevent and/or management of a disease, to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, the term “prophylactically effective amount” of a compound means an amount sufficient to prevent a disease or disorder, or one or more symptoms associated with the disease or disorder, or prevent its recurrence. The term “prophylactically effective amount” can encompass an amount that improves overall prophylaxis or enhances the prophylactic efficacy of another prophylactic agent.

The terms “co-administration” and “in combination with” include the administration of two therapeutic agents (i.e., bexarotene and anti-lipid agent) either simultaneously, concurrently or sequentially with no specific time limits. In one embodiment, both agents are present in the cell or in the patient's body at the same time or exert their biological or therapeutic effect at the same time. In one embodiment, the two therapeutic agents are in the same composition or unit dosage form. In another embodiment, the two therapeutic agents are in separate compositions or unit dosage forms.

The term “unit dosage form(s)” includes: tablets; caplets; capsules, such as soft elastic gelatin capsules; sachets; cachets; troches; lozenges; dispersions; powders; solutions; gels; liquid dosage forms suitable for oral or mucosal administration to a patient, including suspensions (e.g., aqueous or non-aqueous liquid suspensions), emulsions (e.g., oil-in-water emulsions, or a water-in-oil liquid emulsion), solutions, and elixirs; and sterile solids (e.g., crystalline or amorphous solids) that can be reconstituted to provide liquid dosage forms suitable for oral or parenteral administration to a patient. The unit dosage form does not necessarily have to be administered as a single dose.

Bexarotene

Bexarotene (Targretin®) is a member of a subclass of compounds called retinoids. Certain retenoids are believed to selectively activate retinoid X receptors (RXRs). A chemical name for bexarotene is 4-[1-(5,6,7,8-tetrahydro-3,5,5,8,8-pentamethyl-2-naphthalenyl) ethenyl] benzoic acid, and its structural formula is as follows:

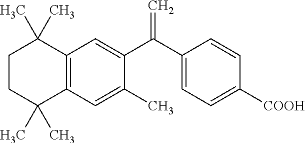

Bexarotene is currently approved for use in the United States as a prescription drug. It is approved for treatment of patients with early-stage cutaneous T-cell lymphoma (CTCL) who have not tolerated other therapies, patients with refractory or persistent early stage CTCL and patients with refractory advanced stage CTCL (see, PDR 2005). Oral bexarotene is currently used in the treatment of all stages of CTCL in patients refractory to at least one prior systemic therapy. Bexarotene gel is used for the treatment of cutaneous lesions in patients with CTCL. Bexarotene is also undergoing studies for use in treating melanoma, non small cell lung cancer and in treatment of women at high genetic risk for breast cancer (see, Wu et al., Cancer Epidemiology, Biomarkers and Prevention (2002), 11, 467-474 and Rigas et al. Oncologist (US) (2005), 10 (1) P22-33). Bexarotene is administered orally in capsule form, or as a gel that is administered directly onto the skin.

Prostate Intraepithelial Neoplasia (PIN)

In any chemoprevention strategy, the availability of histologically recognizable and accepted precancerous lesions constitute an important starting point. For the prostate, a histological marker is a precancerous precursor of prostatic adenocarcinoma, such as prostatic intraepithelial neoplasia (PIN). Two grades of PIN are generally recognized: low-grade PIN (LGPIN) and high-grade PIN (HGPIN). HGPIN is considered to be a precursor to many, if not most, invasive prostate cancers based on clinical, morphologic, histochemical and molecular data. In contrast, the diagnosis of LGPIN is subjective and is not clinically significant.

HGPIN is the most accurate and reliable marker of prostate carcinogenesis and can be used as an acceptable endpoint in prostate chemoprevention trials. HGPIN has a high predictive value as a marker for adenocarcinoma, and its identification warrants repeat biopsy for concurrent or subsequent invasive carcinoma. Further, HGPIN represents unstable epithelia that may progress to prostate cancer, although other precursor abnormalities are also likely to be present. HGPIN is associated with progressive abnormalities of phenotype and genotype that represent a continuum between normal prostatic epithelium and cancer. The presence of HGPIN indicates that abnormalities of cell differentiation and regulatory control have already started, but they may be reversible at this stage. Thus, the development of high grade PIN represents an important step in the progression pathway whereby the normal prostate develops PIN, histological prostate cancer, invasive clinical prostate cancer, and metastases. Most studies suggest that most patients with HGPIN will develop carcinoma within 10 years. HGPIN does not contribute to serum PSA, since unlike prostate cancer, prostate intraepithelial neoplasia has not yet invaded the vasculature of the prostate to leak PSA into the blood stream. Thus, prostate intraepithelial neoplasia may precede even prostate cancer related serum PSA elevations. Treatment of PIN as provided herein can reduce the incidence of prostate cancer, suppress or inhibit the latent prostate cancer and manage prostate carcinogenesis.

Methods used for diagnosis of PIN are known in the art and include, but are not limited to, needle biopsy and measurement of expression of ezrin, a cytoskeleton linker protein that is actively involved in regulating the growth and metastatic capacity of cancer cells (see, Pang et al., Urology. 2004 Mar; 63(3):609-12, Expression of ezrin in prostatic intraepithelial neoplasia). U.S. Pat. No. 6,054,320 provides kits for identification of PIN.

Methods Of Treatment

In one embodiment, provided herein are methods for treatment of prostate intraepithelial neoplasia by administering bexarotene or a pharmaceutically acceptable derivative thereof. Further provided are methods for reducing the risk of developing prostate cancer by administering bexarotene or a pharmaceutically acceptable derivative thereof. Also provided are methods for suppressing or inhibiting latent prostate cancer by administering bexarotene or a pharmaceutically acceptable derivative thereof. In one embodiment, provided herein are methods for preventing prostate carcinogenesis by administering bexarotene or a pharmaceutically acceptable derivative thereof. Further provided are methods for reducing the amount of precancerous precursors of prostate adenocarcinoma lesions by administering bexarotene or a pharmaceutically acceptable derivative thereof. In certain embodiments, methods for treating prostate cancer by administering bexarotene or a pharmaceutically acceptable derivative thereof are provided.

In certain embodiments, the methods provided herein further include administration of other therapeutic agents. Such agents include, but are not limited to anti-lipid agents, such as statins, for example, atorvastatin, fluvastatin, lovastatin, pravastatin, rosouvastatin, and simvastatin; fibrates, such as fenofibrate; and anti-hypothyroid agents such as levothyroxine, liothyronine, liotrix, thyroglobulin, and thyroid.

In certain embodiments, the methods further include administration of a chemopreventive agent known in the art. Exemplary classes of chemopreventive agents that can be used in the methods provided herein include, but are not limited to, selective estrogen receptor modulators (SERMS) and other hormonal agents; nonsteroidal anti-inflammatory drugs (NSAIDS); calcium compounds; glucocorticoids; and retinoids other than bexarotene. Examples of various chemopreventive agents include but are not limited to tamoxifen, aspirin, piroxicam, celecoxib, sulindac, selenium, vitamin E, 2-difluoromethylomithine (DFMO) (also called eflornithine), folic acid, oltipraz, and genistein.

In certain embodiments, methods provided herein are used for human patient population of age ranging from about 20-95 years, from about 30-80 years, and from about 40-60 years. In certain embodiments, methods provided herein are used for human patient population of age ranging from about 45-85 years, from about 50-75 years and from about 50-60 years. In certain embodiment the patient population is of age about 20 years, 25 years, 30 years, 35 years, 40 years, 45 years, 50 years, 50 years, 55 years, 60 years, 60 years, 65 years, 70 years, 75 years, 80 years, 85 years, 90 years or 95 years.

In certain embodiments, bexarotene is administered in an amount ranging from about 75 mg up to about 600 mg per day. In one embodiment, the amount of bexarotene administered is about 100 mg, about 125 mg, about 200 mg, 250 mg, about 300 mg, about 400 mg, about 500 mg, about 600 mg, about 700 mg or about 800 mg per day. In one embodiment, the amount of bexarotene administered is about 150 mg, about 225 mg or about 300 mg per day. In one embodiment, the amount of bexarotene administered is about 225 mg per day.

Pharmaceutical Compositions and Dosage Forms

Pharmaceutical compositions and dosage forms for use in the methods provided herein contain bexarotene or a pharmaceutically acceptable derivative thereof in a pharmaceutically acceptable carrier and in amounts that are useful in the methods provided herein. Such methods include, but are not limited to, treatment of prostate intraepithelial neoplasia, reducing the risk of developing prostate cancer, suppressing or inhibiting latent prostate cancer, preventing prostate carcinogenesis, treating prostate cancer and reducing the amount of precancerous precursors of prostate adenocarcinoma lesions.

Bexarotene for use herein is formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the formulation are prepared using techniques and procedures well known in the art (see, e.g., Ansel Introduction to Pharmaceutical Dosage Forms, Seventh Edition 1999,).

In the compositions, effective concentrations of bexarotene or a pharmaceutically acceptable derivatives is (are) mixed with a suitable pharmaceutical carrier or vehicle. Bexarotene may be derivatized as the corresponding salts, esters, enol ethers or esters, acids, bases, solvates, hydrates or prodrugs prior to formulation, as described above. The concentration of bexarotene in the compositions are effective for delivery of an amount, upon administration, that treats, prevents, or ameliorates one or more of the symptoms of conditions associated with prostate intraepithelial neoplasia or prostate cancer.

Typically, the compositions are formulated for single dosage administration. To formulate a composition, the weight fraction of bexarotene is dissolved, suspended, dispersed or otherwise mixed in a selected vehicle at an effective concentration such that the treated condition is relieved or ameliorated. Pharmaceutical carriers or vehicles suitable for administration of bexarotene include any such carriers known to those skilled in the art to be suitable for the particular mode of administration.

In addition, bexarotene may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 5,571,534. Briefly, liposomes such as multilamellar vesicles (MLV's) may be formed by drying down egg phosphatidyl choline and brain phosphatidyl serine (7:3 molar ratio) on the inside of a flask. A solution of bexarotene provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Bexarotene is included in the pharmaceutically acceptable carrier in an amount sufficient to exert desired effect in the patient treated. The therapeutically effective concentration may be determined empirically by testing bexarotene in in vitro and in vivo systems known to one of skill in the art and then extrapolated therefrom for dosages for humans.

The concentration of bexarotene in the pharmaceutical composition will depend on absorption, inactivation and excretion rates of bexarotene, the dosage schedule, and amount administered as well as other factors known to those of skill in the art.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 20th ed., Mack Publishing, Easton Pa. (2000).

Typically a therapeutically effective dosage should produce a serum concentration of active ingredient of from about 0.1 ng/ml to about 50-100 μg/ml. Pharmaceutical dosage unit forms are prepared to provide from about 25 mg to about 500 mg and from about 50 to about 400 mg, or from about 75 up to about 300 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the compositions provided herein.

Pharmaceutically acceptable derivatives include salts, esters, hydrates, solvates and prodrug forms. The derivative is selected such that its pharmacokinetic properties are superior to bexarotene itself.

Thus, effective concentrations or amount of bexarotene or its pharmaceutically acceptable derivative is mixed with a suitable pharmaceutical carrier or vehicle for systemic, topical or local administration to form the pharmaceutical composition. Bexarotene is included in an amount effective for treating or preventing prostate intraepithelial neoplasia or prostate cancer or disorders associated therewith as described herein.

The compositions are intended to be administered by a suitable route, including orally, parenterally, rectally, topically and locally. Bexarotene or a pharmaceutically acceptable derivative thereof is typically formulated and administered in unit-dosage forms such as tablets, capsules, pills, powders, granules, sterile parenteral solutions or suspensions, and oral solutions or suspensions, and oil-water emulsions containing suitable quantities of the active ingredient or multiple-dosage forms. Unit-dose forms as used herein refers to physically discrete units suitable for human and animal subjects and packaged individually as is known in the art. Each unit-dose contains a predetermined quantity of bexarotene sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Particular lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

a. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art. Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000)

In certain embodiments, the formulations are solid dosage forms, such as capsules or tablets. The tablets, pills, capsules, troches and the like can contain any of the following ingredients, or conjugates of a similar nature: a binder, a filler, a diluent; a disintegrating agent; a lubricant; a glidant; a sweetening agent; and a flavoring agent. Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, or from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SM (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

If oral administration is desired, bexarotene could be provided in a composition that is formulated as enteric coating tablets, sugar-coated tablets, film-coated tablets or multiple compressed tablets. Enteric coating tablets protect the active ingredient from the acidic environment of the stomach. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges. The composition may also be formulated in combination with an antacid or other such ingredient.

When the dosage unit form is a capsule, it can contain, in addition to material of the above type, a liquid carrier such as a fatty oil. In a gelatin capsule, the solution or suspension containing bexarotene, in for example propylene carbonate, vegetable oils or triglycerides, can be encapsulated in the capsule.

The active ingredient can also be mixed with other active materials which do not impair the desired action, or with materials that supplement the desired action, such as antacids, H2 blockers, and diuretics. Higher concentrations, up to about 98% by weight of the active ingredient may be included.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative.

An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia.

Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

The pharmaceutical compositions containing active ingredients in micellar form can be prepared as described in U.S. Pat. No. 6,350,458. Such pharmaceutical compositions are particularly effective in oral, nasal and buccal applications.

In certain embodiments, formulations include, but are not limited to, those containing bexarotene, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In certain embodiments, bexarotene is formulated as an oral capsule containing about 75 mg, about 100 mg, about 250 mg, about 200 mg, about 225 mg, about 250 mg, about 300 mg, about 400 mg, about 500 mg or about 600 mg, about 700 mg or about 800 mg of the active ingredient. The capsule can contain inactive ingredients, such as polyethylene glycol 400, polysorbate 20, povidone, and butylated hydroxyanisole. The capsule shell can contain gelatin, sorbitol special glycerin blend and titanium dioxide.

b. Controlled Release Dosage Form

Bexarotene can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with bexarotene. Thus, the methods and compositions provided herein encompass single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

c. Parenteral Administration

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of bexarotene is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active ingredient is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, or more than 1% w/w of bexarotene to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Bexarotene may be suspended in micronized or other suitable form or may be derivatized to produce a more soluble active product or to produce a prodrug. The form of the resulting mixture depends upon a number of factors, including the intended mode of administration and the solubility of bexarotene in the selected carrier or vehicle. The effective concentration is sufficient for ameliorating the symptoms of the condition and may be empirically determined.

d. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving the active ingredient, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of bexarotene. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg or about 9-30 mg of lyophilized powder, is added per mL of sterile water or other suitable carrier. The precise amount can be empirically determined.

e. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Bexarotene may be formulated for local or topical application, such as for topical application to the skin and mucous membranes, in the form of gels, creams, and lotions. Topical administration is contemplated for transdermal delivery and also for administration mucosa, or for inhalation therapies.

f. Compositions for Other Routes of Administration

Other routes of administration, such as topical application, transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

In certain embodiments, the formulation is a gel for rectal administration. In certain embodiments, the rectal gel can contain dehydrated alcohol, polyethylene glycol 400, hydroxypropyl cellulose, and bulylated hydroxytoluene in appropriate amounts.

Articles of Manufacture

Bexarotene for use in the methods provided herein can be packaged as an article of manufacture using packaging materials well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

Kits

In certain embodiments, bexarotene is administered in combination with other therapeutic agents provided herein. The other therapeutic agents may or may not be administered to a patient at the same time or by the same route of administration. Provided herein, therefore, are kits which, when used by the medical practitioner, can simplify the administration of appropriate amounts of active ingredients to a patient

A typical kit includes a container containing a dosage form of bexarotene or a pharmaceutically acceptable derivative thereof and a container containing one or more other therapeutic agent(s) described elsewhere herein. Such other therapeutic agents include, but are not limited to anti-lipid agents, anti-hypothyroid agents, and chemopreventive agents.

Kits provided herein further include devices that are used to administer the active ingredients. Examples of such devices include, but are not limited to, syringes, needle-less injectors drip bags, patches, and inhalers.

Kits provided herein further include cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Evaluation of the Activity of Bexarotene

Standard physiological, pharmacological and biochemical procedures are available are known to one of skill in the art to test the efficacy of bexarotene in the methods provided herein. In vitro and in vivo assays that can be used to evaluate biological activity contemplated herein. An exemplary in vivo mouse model Transgenic Adenocarcinoma Mouse Prostate (TRAMP) (see, Greenberg et al., A prostate cancer in a transgenic mouse, Proc. Natl Acad. Sci. USA, 1995, Vol. 92, pages 3439-3443) to determine the mechanisms of initiation and promotion of prostate cancer and to test the effectiveness of potential chemopreventive agent is described in U.S. Pat. No. 6,410,043. The TRAMP transgenic mouse model or similar such models can be used to test the effectiveness bexarotene or pharmaceutically active derivatives thereof in treatment of prostate intraepithelial neoplasia, reducing the risk of developing prostate cancer, suppressing or inhibiting latent prostate cancer, preventing prostate carcinogenesis, treating prostate cancer and reducing the amount of precancerous precursors of prostate adenocarcinoma lesions. These mice progressively develop prostate epithelial hyperplasia, PIN, and then prostate cancer within a short period (<17 weeks). Other procedures known in the art can be used to test the effectiveness of bexarotene in the methods provided herein can also be employed.

### EXAMPLES

### Example 1

A 70 year old male was diagnosed as having High Grade PIN. The diagnosis was based upon 12 biopsies which were done in conjunction with ultrasound localization of the biopsy site, with careful recording of there anatomical position. The patient was started on Bexarotene 225 mg per day. The drug was given as a single dose once daily. The drug was administered for 30 doses with two 4 day interruptions because of the development of an inguinal monilia infections treated appropriately each time. During the period of therapy the patient was on Atorvastatin 20 mg daily. During the therapy, total cholesterol was 183 mg with a reference range <200 mg/dL, triglycerides were 207 with normal being less than 150 mg/dL, thyroid stimulation hormone (TSH) was 0.07 with the reference range 0.4-5.5 mIU/L, T4 total was 7.4 with the normal range 4.5-12.0 mcg/dL. The Hepatic Function Panel was normal as was the complete blood count (CBC). Two weeks following the last dose of Bexarotene the prostate was rebiopsied with 12 biopsies taken. Six of the biopsies were taken from the putative area, the rest in an enlarging target like pattern. The repeat biopsy was negative for the presence of PIN.

All of the references cited herein are incorporated by reference in their entirety. While the invention has been described with respect to the particular embodiments, it will be apparent to those skilled in the art that various changes and modifications may be made without departing from the spirit and scope of the claimed subject matter as recited by the appended claims.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A method for treatment of prostate intraepithelial neoplasia comprising administering therapeutically effective amount bexarotene or a pharmaceutically acceptable derivative thereof to a patient in need of the treatment.

2. A method for reducing a risk of developing prostate cancer comprising administering bexarotene or a pharmaceutically acceptable derivative thereof to a patient in need of reducing the risk.

3. A method for treatment for suppressing a latent prostate cancer comprising administering bexarotene or a pharmaceutically acceptable derivative thereof to a patient in need of the treatment.

4. A method for preventing prostate carcinogenesis comprising administering bexarotene or a pharmaceutically acceptable derivative thereof to a patient in need for preventing prostate carcinogenesis.

5. A method for treating prostate cancer by administering bexarotene or a pharmaceutically acceptable derivative thereof to a patient in of the treatment.

6. A method for reducing an amount of a precancerous precursor of prostate adenocarcinoma lesion comprising administering bexarotene or a pharmaceutically acceptable derivative thereof to a patient in need of the treatment.

7. A method for managing prostate intraepithelial neoplasis by administering bexarotene or a pharmaceutically acceptable derivative thereof to a patient in need of managing prostate intraepithelial neoplasis.

8. The method of claim 1 further comprising administering an anti-lipid agent.

9. The method of claim 8, wherein the anti-lipid agent is a statin or a fibrate.

10. The method of claim 9, wherein the statin is atorvastatin, fluvastatin, lovastatin, pravastatin, rosouvastatin, or simvastatin.

11. The method of claim 9, wherein the statin is atorvastatin.

12. The method of claim 9, wherein the fibrate is fenofibrate.

13. The method of claim 1 further comprising administering an anti-hypothyroid agent.

14. The method of claim 13, wherein the anti-hypothyroid agent is levothyroxine, liothyronine, liotrix, thyroglobulin, or thyroid.

15. The method of claim 1 further comprising administering a chemopreventive agent.

16. The method of claim 15, wherein the chemopreventive agent is a selective estrogen receptor modulator, nonsteroidal anti-inflammatory drug, calcium compound, glucocorticoid or retinoid other than bexarotene.

17. The method of claim 16, wherein the chemopreventive agent is tamoxifen, aspirin, piroxicam, celecoxib, sulindac, selenium, vitamin E, 2-difluoromethylornithine, folic acid, oltipraz, or genistein.

18. The method of claim 1, wherein the bexarotene is administered in a single dose.

19. The method claim 1, wherein the bexarotene is administered once daily.

20. The method of claim 1, wherein the bexarotene is administered in an amount from about 75 mg up to about 600 mg/day.

21. The method of claim 1, wherein the amount of bexarotene administered is 300 mg/day.

22. The method of claim 1, wherein the amount of bexarotene administered is 225 mg/day.

23. The method of claim 1, where the prostate intraepithelial neoplasia comprises HGPIN.

24. The method of claim 1, wherein the patient is an adult male.

25. The method of claim 1, wherein the patient is in an age group range from about 20 years to about 80 years.

26. The method of claim 1, wherein the patient is about 55 years of age.

27. The method of claim 1, wherein the patient is about 65 years of age.

28. The method of claim 1, wherein the bexarotene is administered as an oral formulation.

29. The method of claim 28, wherein the oral formulation is a capsule.

30. The method of claim 1, wherein the bexarotene is administered as a rectal formulation.

31. The method of claim 30, wherein the rectal formulation is a gel.

32-38. (canceled)
